(19) United States
(12) Patent Application Publication
(10) Pub. No.: US 2026/0108451 A1
(43) Pub. Date: Apr. 23, 2026

(54) COMPOSITIONS FOR THE MANAGEMENT OF HIRSUTISM

(71) Applicants: Anju Majeed, Bangalore (IN); Shaheen Majeed, Payson, UT (US); Sabinsa Corporation, East Windsor, NJ (US)

(72) Inventors: Muhammed Majeed, Bangalore (IN); Kalyanam Nagabhushanam, East Windsor, NJ (US); Sarang Bani, Bangalore (IN); Anjali Pandey, Bangalore (IN); Lakshmi Mundkur, Bangalore (IN)

(21) Appl. No.: 19/117,020

(22) PCT Filed: Sep. 29, 2023
(86) PCT No.: PCT/US2023/075565

§ 371 Date: Mar. 28, 2025

(30) Foreign Application Priority Data
Sep. 29, 2022 (IN) 202241055913

(51) Int. Cl. A61K 8/92; A61K 8/35; A61K 8/69; A61K 8/9789; A61Q 7/02

(52) U.S. Cl. CPC A61K 8/922; A61K 8/35; A61K 8/69; A61K 8/9789; A61Q 7/02; A61K 2800/5922; A61K 2800/782

(57) ABSTRACT

The present invention discloses Anti-hirsutism compositions, use and methods for managing hirsutism in a subject using the composition comprising turmeric oil standardized to contain Aromatic Turmerone (ar-Turmerone), Eflornithine; Nigella sativa extract; Oroxylum indicum extract; and Licorice extract either individually or combinations thereof. The invention also discloses use of the above composition in inhibiting the activity of ornithine decarboxylase and 5-α-reductase.

### CROSS REFERENCE TO RELATED APPLICATIONS

This is a National Phase application of PCT application no. PCT/US23/75565, filed on 29 Sep. 2023, claiming priority to Indian Provisional Application No. 202241055913 filed on 29 Sep. 2022, the contents of which are incorporated herein by reference.

### FIELD OF THE INVENTION

The invention in general relates to a method and composition of modulating hair growth and managing hirsutism in a subject. More specifically, the invention relates to a composition comprising Aromatic Turmerone (AR-Turmerone) individually or in combination with different plant actives for the management of hirsutism.

### BACKGROUND OF THE INVENTION

Hirsutism, derived from the Latin word “hisutus” meaning shaggy, rough and bristlyis a cosmetic and psychosocial problem due to the presence of excessive hair growth in androgen dependent areas. Hirsutism results from excess production of androgens, often from ovarian or adrenal sources. Hirsutism is directly affected by the androgen levels and the sensitivity of hair follicles to androgen. Excess androgen levels can cause fine, “vellous” hair follicles to produce larger, darker “terminal” hair growth in most androgen-sensitive areas mainly upper lip, chin, chest, back, and upper abdominal area. Hirsutism can be underlying endocrine disorder in Cushing's disease, Hyperthecosis, Adrenal tumors, Ovarian tumors, insulin resistance, congenital adrenal hyperplasia, polycystic ovarian syndrome, and Idiopathic hirsutism, (Int J Women's Dermatol. 2015 June; 1 (2): 90-94. 2015). Mild or moderate hirsutism may not require treatment, the Endocrine society guidelines suggest “patient important hirsutism” to underline the customized treatment considering patient preference and severity of the condition.

There are multiple treatment options for Hirsutism such as direct hair removal methods like electrolysis or laser hair removal, shaving, plucking, topical creams, waxing, threading, or bleaching offering temporary cosmetic ways to manage Hirsutism. Laser/photo epilation therapy is preferred choice for hair removal therapy but for those desirous of rapid initial response adding Eflornithine was suggested (Martin et al. J Clin Endocrinol Metab. 93 2008, 1105-1120). Further, in five independent trials it was identified anti-androgen therapy using spironolactone, finasteride, and flutamide decreased hirsutism with no differences among the medication compared to placebo. (Swiglo et al. 2008 J. Clin. Endocrinol. Metab. 93 1153-1160). The 2008 Endocrine Society guidelines discourages antiandrogen monotherapy unless effective contraceptive was used, due to teratogenic effects. In general, these drugs have major side effects some include dryness of skin or scalp hair, liver enzyme abnormalities, fatal hepatotoxicity, adrenal insufficiency, loss of libido, teratogenicity (Mihailidis et al. Int J Women's Dermatol. 1 (2015) 90-94).

Eflornithine hydrochloride 13.9% (Vaniqa®) acts by irreversibly inhibiting L-ornithine decarboxylase, an enzyme important for controlling hair growth and proliferation (Balfour, et. al, Am. J Clin Dermatol 2 (3) 2001, 197-201). Controlled trials have reported a significant reduction in hair growth when a combination of laser therapy with Eflornithine was used in contrast to laser therapy with placebo cream, especially early in the treatment course (Hamzavi et al. J. Am. Acad Dermatol. 57, 2007, 54-59). In a study involving mice, pretreatment of skin with microneedles before topical application of Eflornithine showed enhanced ability of Eflornithine in inhibiting hair growth (Amit kumar et al., Drug Deliv. 23 (5) 2016, 1495-1501). There are no remedies to completely stop the unwanted hair growth and one needs to wait 6 months or more to witness reduced hair growth. Preferably, Eflornithine cream must be used for a long term to maintain the improvement. Also, topical application may not suit every skin type, sensitive skin type may get side effects like stinging, irritation, dryness, and redness or swollen patches of skin that are reddened and contain buried hair at the application site.

Turmeric has been traditionally used for removing excessive and unwanted hairs from face and other parts of the body. Typically, the oil derived from turmeric rhizomes have bene used in the treating and managing symptoms of hirsutism. Turmeric oil is standardized to contain 44.72±2.14% α-turmerone, 20.77±0.63% β-turmerone, and 34.51±2.69% ar-turmerone (Takemoto et al., Sci Rep. 2022; 12: 11039). However, the actives responsible for the anti-hirsutistic activity is still not identified. There still exists an unmet need for alternative treatment which not only provides effective treatment but also safe with no side effects. The present invention solves the above need to disclosing a composition comprising Aromatic Turmerone; Eflornithine; Nigella sativa extract; Oroxylum indicum extract; and Licorice extract, individually or in combination for the management of hirsutism.

It is the principal object of the invention to disclose a composition comprising turmeric oil standardized to contain Aromatic Turmerone; Eflornithine; Nigella sativa extract; Oroxylum indicum extract; and Licorice extract individually or in combination for use in the management of hirsutism.

It is another object of the invention to disclose a composition comprising turmeric oil standardized to contain Aromatic Turmerone; Eflornithine; Nigella sativa extract; Oroxylum indicum extract; and Licorice extract individually or in combination for use inhibiting enzymes involved in modulating hair growth in a subject.

The present invention solves the above disclosed objects and provides further related advantages.

### SUMMARY OF THE INVENTION

In a most preferred embodiment, the invention discloses an Anti-Hirsutism composition comprising turmeric oil standardized to contain Aromatic Turmerone (Ar-Turmerone) as an active agent, wherein the composition further comprises active agent selected from the group consisting of Eflornithine, Nigella sativa extract, Oroxylum indicum extract, Licorice extract or combinations thereof.

In another preferred embodiment, the invention discloses a composition comprising turmeric oil standardized to contain Aromatic Turmerone (Ar-Turmerone); Eflornithine; Nigella sativa extract; Oroxylum indicum extract; and Licorice extract; for use either individually or in combination in the management of hirsutism in a subject.

In another preferred embodiment, the invention discloses a composition comprising turmeric oil standardized to contain Ar-Turmerone; Eflornithine; Nigella sativa extract; Oroxylum indicum extract; and Licorice extract; for use either individually or in combination in inhibiting enzymes involved in modulating hair growth in a subject.

In another preferred embodiment, the invention discloses a method of management of hirsutism in a subject, comprising step of a) identifying the subject in need of hirsutism management; and b) administering a composition comprising turmeric oil standardized to contain Ar-Turmerone; Eflornithine; Nigella sativa extract; Oroxylum indicum extract; and Licorice extract; either individually or in combination.

In another preferred embodiment, the invention discloses a method of inhibiting enzymes involved in modulating hair growth in subject, comprising step of bringing into contact a composition comprising turmeric oil standardized to contain Ar-Turmerone; Eflornithine; Nigella sativa extract; Oroxylum indicum extract; and Licorice extract; either individually or in combination with the said subject, to bring about reduction in hair growth.

Other features and advantages of the present invention will become apparent from the following detailed description, which illustrate, by way of examples, the principle of the invention.

### BRIEF DESCRIPTION OF DRAWINGS

FIG. 1 shows the flow cytometric detection of the expression of ornithine decarboxylase enzyme treated with Ar-Turmerone (turmeric oil) and eflornithine.

FIG. 2 shows the change in expression of ornithine decarboxylase enzyme in cells treated with Ar-Turmerone (turmeric oil) and eflornithine.

FIG. 3 shows the change in expression of 5-α reductase enzyme in cells treated with AR turmerone (turmeric oil).

### DEFINITIONS

All the terms used in this application carry ordinary meaning as known in the prior art unless otherwise specified. A few other specific definitions used in this invention are explained below, which apply throughout this specification.

Therapeutically managing, management. modulating or treating refers to a condition of effectively ameliorating conditions disclosed in the invention.

The “% w/w” of actives represent purity of the active constituents.

### DESCRIPTION OF PREFERRED EMBODIMENTS

In the most preferred embodiment of the invention, the invention discloses anti-hirsutism composition comprising turmeric oil standardized to contain Ar-Turmerone as an active agent, wherein the composition further comprises agent selected from the group consisting of Eflornithine; Nigella sativa extract; Oroxylum indicum extract; and Licorice extract or combinations thereof. Alternatively, in yet another preferred embodiment of the invention, extract from Oroxylum indicum or Nigella sativa can be either individually used or as a combination selected from the group consisting of Eflornithine, turmeric oil standardized to contain Ar-Turmerone, or combinations thereof. In a related aspect of the invention, the combinations are selected from the group consisting of turmeric oil standardized to contain Ar-Turmerone and Eflornithine, turmeric oil standardized to contain Ar-Turmerone and Nigella sativa extract, turmeric oil standardized to contain Ar-Turmerone and Oroxylum indicum extract, turmeric oil standardized to contain Ar-Turmerone and Licorice extract, or turmeric oil standardized to contain Ar-Turmerone, Oroxylum indicum extract and Nigella sativa extract. In another related aspect, the turmeric oil is standardized to contain 20-50% w/w aromatic turmerone and 50-90% w/w total turmerones. In yet another related aspect, the Nigella sativa extract is standardized to contain not less than 2% w/w thymoquinone, 0.01-10% w/w thymohydroquinone, 15-95% w/w fatty acids, 0.001-3% w/w α-hederin, 0.1-4% w/w rosmarinic acid and 0.2-2% w/w piperine. In another aspect of the invention the Oroxylum indicum extract is standardized to contain 10-15% w/w oroxylin A, 10-25% w/w baicalein, and 2-10% w/w chrysin. In related aspect of the invention, Licorice extract is standardized to contain 40-50% w/w glabridin. In related aspect of the invention, the composition ameliorates the symptoms of hirsutism by inhibiting enzymes related to hair growth, wherein the enzymes are selected from the group consisting of ornithine decarboxylase and 5-α-reductase. In another related aspect of the invention, the concentration of actives in the composition are in the range 1 μg/ml to 150 μg/ml. In yet another related aspect of the invention, the individual extracts in the combination are present in amounts of not less than 10% w/w of total composition. In all aspects of the invention, the composition further comprises of stabilizing agents, bioavailability enhancers, antioxidants, cosmeceutically accepted excipients, and topically administered in the form of cream, lotion, oil, serum, gel or emulsion.

In another preferred embodiment, the invention discloses a composition comprising turmeric oil standardized to contain Aromatic Turmerone; Eflornithine; Nigella sativa extract; Oroxylum indicum extract; and Licorice extract for use either individually or in combination for in the management of hirsutism in a subject. In a related aspect of the invention, management of hirsutism is brought about by inhibiting enzymes involved in modulating hair growth, thereby decreasing hair growth in the subject. In a related aspect of the invention, the combinations are selected from the group consisting of turmeric oil standardized to contain Ar-Turmerone and Eflornithine, turmeric oil standardized to contain Ar-Turmerone and Nigella sativa extract, turmeric oil standardized to contain Ar-Turmerone and Oroxylum indicum extract, turmeric oil standardized to contain Ar-Turmerone and Licorice extract, or turmeric oil standardized to contain Ar-Turmerone, Oroxylum indicum extract and Nigella sativa extract. In yet another related aspect of the invention, the turmeric oil is standardized to contain 20-50% w/w Ar-Turmerone and 50-90% w/w total turmerones. In another related aspect, the Nigella sativa extract is standardized to contain not less than 2% w/w thymoquinone, 0.01-10% w/w thymohydroquinone, 15-95% w/w fatty acids, 0.001-3% w/w α-hederin, 0.1-4% w/w rosmarinic acid and 0.2-2% w/w piperine. In another aspect of the invention, the Oroxylum indicum extract is standardized to contain 10-15% w/w oroxylin A, 10-25% w/w baicalein, and 2-10% w/w chrysin. In an aspect, the Licorice extract is standardized to contain 40-50% w/w glabridin. In a further aspect, the enzymes are selected from the group consisting of ornithine decarboxylase and 5-α-reductase. In yet another further aspect, the concentration of actives in the composition are in the range 1 μg/ml to 150 μg/ml. In another aspect, the individual extracts in the combination are present in amounts of not less than 10% w/w of total composition. In all aspects of the invention, the subject is a mammal. In all aspects of the invention, the composition further comprises of stabilizing agents, bioavailability enhancers, antioxidants, cosmeceutically accepted excipients and topically administered in the form of cream, lotion, oil, serum, gel or emulsion.

In another preferred embodiment of the invention, discloses a composition comprising turmeric oil standardized to contain Ar-Turmerone; Eflornithine; Nigella sativa extract; Oroxylum indicum extract; and Licorice extract; for use either individually or in combination in inhibiting enzymes involved in modulating hair growth in a subject. In a related aspect of the invention, the combinations are selected from the group consisting of turmeric oil standardized to contain Ar-Turmerone and Eflornithine, turmeric oil standardized to contain Ar-Turmerone and Nigella sativa extract, turmeric oil standardized to contain Ar-Turmerone and Oroxylum indicum extract, turmeric oil standardized to contain Ar-Turmerone and Licorice extract, or turmeric oil standardized to contain Ar-Turmerone, Oroxylum indicum extract and Nigella sativa extract. In yet another related aspect, the turmeric oil is standardized to contain 20-50% w/w Ar-Turmerone and 50-90% w/w total turmerones. In another related aspect, the Nigella sativa extract is standardized to contain not less than 2% w/w thymoquinone, 0.01-10% w/w thymohydroquinone, 15-95% w/w fatty acids, 0.001-3% w/w α-hederin, 0.1-4% w/w rosmarinic acid and 0.2-2% w/w piperine. In another aspect of the invention, the Oroxylum indicum extract is standardized to contain 10-15% w/w oroxylin A, 10-25% w/w baicalein, and 2-10% w/w chrysin. In another aspect of the invention, the Licorice extract is standardized to contain 40-50% w/w glabridin. In further aspect of the invention, the enzymes are selected from the group consisting of ornithine decarboxylase and 5-α-reductase. In another aspect, inhibiting enzymes involved in hair growth is effective in the management of hirsutism. In yet another further aspect, the concentration of actives in the composition are in the range 1 μg/ml to 150 μg/ml. In another aspect, the individual extracts in the combination are present in amounts of not less than 10% w/w of total composition. In all aspects of the invention, the subject is a mammal or human cell. In all aspects of the invention, the composition further comprises of stabilizing agents, bioavailability enhancers, antioxidants, cosmeceutically accepted excipients and topically administered in the form of cream, lotion, oil, serum, gel or emulsion

In another preferred embodiment, the invention discloses use of a composition comprising turmeric oil standardized to contain Aromatic Turmerone; Eflornithine; Nigella sativa extract; Oroxylum indicum extract; and Licorice extract either individually or in combination in the management of hirsutism in a subject. In a related aspect, management of hirsutism is brought about by inhibiting enzymes involved in modulating hair growth, thereby decreasing hair growth in the subject. In a related aspect of the invention, the combinations are selected from the group consisting of turmeric oil standardized to contain Ar-Turmerone and Eflornithine, turmeric oil standardized to contain Ar-Turmerone and Nigella sativa extract, turmeric oil standardized to contain Ar-Turmerone and Oroxylum indicum extract, turmeric oil standardized to contain Ar-Turmerone and Licorice extract, or turmeric oil standardized to contain Ar-Turmerone, Oroxylum indicum extract and Nigella sativa extract. In yet another related aspect of the invention, the turmeric oil is standardized to contain 20-50% w/w Ar-Turmerone and 50-90% w/w total turmerones. In another related aspect, the Nigella sativa extract is standardized to contain not less than 2% w/w thymoquinone, 0.01-10% w/w thymohydroquinone, 15-95% w/w fatty acids, 0.001-3% w/w α-hederin, 0.1-4% w/w rosmarinic acid and 0.2-2% w/w piperine. In another aspect of the invention, the Oroxylum indicum extract is standardized to contain 10-15% w/w oroxylin A, 10-25% w/w baicalein, and 2-10% w/w chrysin. In an aspect of the invention, the Licorice extract is standardized to contain 40-50% w/w glabridin. In further aspect of the invention, the enzymes are selected from the group consisting of ornithine decarboxylase and 5-α-reductase. In yet another aspect of the invention, the concentration of actives in the composition are in the range 1 μg/ml to 150 μg/ml. In another aspect of the invention, the individual extracts in the combination are present in amounts of not less than 10% w/w of total composition. In all aspects of the invention, the subject is a mammal. In all aspects of the invention, the composition further comprises of stabilizing agents, bioavailability enhancers, antioxidants, cosmeceutically accepted excipients and topically administered in the form of cream, lotion, oil, serum, gel or emulsion.

In another preferred embodiment of the invention, discloses a use of a composition comprising turmeric oil standardized to contain Ar-Turmerone; Eflornithine; Nigella sativa extract; Oroxylum indicum extract; and Licorice extract either individually or in combination in inhibiting enzymes involved in modulating hair growth in a subject. In a related aspect of the invention, the combinations are selected from the group consisting of turmeric oil standardized to contain Ar-Turmerone and Eflornithine, turmeric oil standardized to contain Ar-Turmerone and Nigella sativa extract, turmeric oil standardized to contain Ar-Turmerone and Oroxylum indicum extract, turmeric oil standardized to contain Ar-Turmerone and Licorice extract, or turmeric oil standardized to contain Ar-Turmerone, Oroxylum indicum extract and Nigella sativa extract. In yet another related aspect, the turmeric oil is standardized to contain 20-50% w/w Ar-Turmerone and 50-90% w/w total turmerones. In another related aspect, the Nigella sativa extract is standardized to contain not less than 2% w/w thymoquinone, 0.01-10% w/w thymohydroquinone, 15-95% w/w fatty acids, 0.001-3% w/w α-hederin, 0.1-4% w/w rosmarinic acid and 0.2-2% w/w piperine. In another aspect of the invention, the Oroxylum indicum extract is standardized to contain 10-15% w/w oroxylin A, 10-25% w/w baicalein, and 2-10% w/w chrysin. In an aspect of the invention, the Licorice extract is standardized to contain 40-50% w/w glabridin. In further aspects of the invention, the enzymes are selected from the group consisting of ornithine decarboxylase and 5-α-reductase. In another aspect of the invention, inhibiting enzymes involved in hair growth is effective in the management of hirsutism. In yet another further aspect, the concentration of actives in the composition are in the range 1 μg/ml to 150 μg/ml. In another aspect, the individual extracts in the combination are present in amounts of not less than 10% w/w of total composition. In all aspects of the invention, the subject is a mammal or human cell. In all aspects of the invention, the composition further comprises of stabilizing agents, bioavailability enhancers, antioxidants, cosmeceutically accepted excipients and topically administered in the form of cream, lotion, oil, serum, gel or emulsion.

In another preferred embodiment, the invention discloses a method of management of hirsutism in a subject, comprising step of a) identifying the subject in need of hirsutism management and b) administering a composition comprising turmeric oil standardized to contain Ar-Turmerone; Eflornithine; Nigella sativa extract; Oroxylum indicum extract; and Licorice extract; either individually or in combination. In a related aspect of the invention, management of hirsutism is brought about by inhibiting enzymes involved in modulating hair growth, thereby decreasing hair growth in the subject. In a related aspect of the invention, the combinations are selected from the group consisting of turmeric oil standardized to contain Ar-Turmerone and Eflornithine, turmeric oil standardized to contain Ar-Turmerone and Nigella sativa extract, turmeric oil standardized to contain Ar-Turmerone and Oroxylum indicum extract, turmeric oil standardized to contain Ar-Turmerone and Licorice extract, or turmeric oil standardized to contain Ar-Turmerone, Oroxylum indicum extract and Nigella sativa extract. In yet another related aspect of the invention, the turmeric oil is standardized to contain 20-50% w/w aromatic turmerone and 50-90% w/w total turmerones. In another related aspect, the Nigella sativa extract is standardized to contain not less than 2% w/w thymoquinone, 0.01-10% w/w thymohydroquinone, 15-95% w/w fatty acids, 0.001-3% w/w α-hederin, 0.1-4% w/w rosmarinic acid and 0.2-2% w/w piperine. In another aspect of the invention, the Oroxylum indicum extract is standardized to contain 10-15% w/w oroxylin A, 10-25% w/w baicalein, and 2-10% w/w chrysin. In an aspect of the invention, the Licorice extract is standardized to contain 40-50% w/w glabridin. In further aspects of the invention, the enzymes are selected from the group consisting of ornithine decarboxylase and 5-α-reductase. In yet another further aspect, the concentration of actives in the composition are in the range 1 μg/ml to 150 μg/ml. In another aspect, the individual extracts in the combination are present in amounts of not less than 10% w/w of total composition. In all aspects of the invention, the subject is a mammal. In further aspects of the invention, the composition further comprises of stabilizing agents, bioavailability enhancers, antioxidants, cosmeceutically accepted excipients and topically administered in the form of cream, lotion, oil, serum, gel or emulsion.

In another preferred embodiment of the invention, disclosed is a method of inhibiting enzymes involved in modulating hair growth in a subject, comprising step of bringing into contact a composition comprising turmeric oil standardized to contain Ar-Turmerone; Eflornithine; Nigella sativa extract; Oroxylum indicum extract; and Licorice extract either individually or in combination with the said subject, to bring about reduction in hair growth. In a related aspect of the invention, the combinations are selected from the group consisting of turmeric oil standardized to contain Ar-Turmerone and Eflornithine, turmeric oil standardized to contain Ar-Turmerone and Nigella sativa extract, turmeric oil standardized to contain Ar-Turmerone and Oroxylum indicum extract, turmeric oil standardized to contain Ar-Turmerone and Licorice extract, or turmeric oil standardized to contain Ar-Turmerone, Oroxylum indicum extract and Nigella sativa extract. In yet another related aspect of the invention, the turmeric oil is standardized to contain 20-50% w/w aromatic turmerone and 50-90% w/w total turmerones. In another related aspect of the invention, the Nigella sativa extract is standardized to contain not less than 2% w/w thymoquinone, 0.01-10% w/w thymohydroquinone, 15-95% w/w fatty acids, 0.001-3% w/w α-hederin, 0.1-4% w/w rosmarinic acid and 0.2-2% w/w piperine. In another aspect of the invention, the Oroxylum indicum extract is standardized to contain 10-15% w/w oroxylin A, 10-25% w/w baicalein, and 2-10% w/w chrysin. In an aspect of the invention, the Licorice extract is standardized to contain 40-50% w/w glabridin. In further aspect of the invention, the enzymes are selected from the group consisting of ornithine decarboxylase and 5-α-reductase. In another aspect of the invention, inhibiting enzymes involved in hair growth is effective in the management of hirsutism. In yet another further aspect of the invention, the concentration of actives in the composition are in the range 1 μg/ml to 150 μg/ml. In another aspect of the invention, the individual extracts in the combination are present in amounts of not less than 10% w/w of total composition. In all aspects of the invention, the subject is a mammal or human cell. In further aspects of the invention, the composition further comprises of stabilizing agents, bioavailability enhancers, antioxidants, cosmeceutically accepted excipients and topically administered in the form of cream, lotion, oil, serum, gel or emulsion.

The following illustrative examples further discloses the preferred embodiments in detail

### EXAMPLES

### Example 1: Extracts

The turmeric oil standardized to contain Ar-turmerones (herein referred to as Ar-Turmerone), eflornithine and licorice extracts are standardized using commercially available methods or are sourced from Sami Sabinsa Group Limited, Bangalore, India. Process for the isolation of thymohydroquinone and other actives and standardization of extracts from Nigella Sativa is disclosed in U.S. Pat. No. 10,945,969 B2. Similarly, isolation of bioactive from Oroxylum indicum is disclosed in U.S. Pat. No. 10,555,982 B2.

### Example 2: Cell Culture

Human Follicle Dermal Papilla Cells (HFDPC) were cultured in follicle dermal papilla cell growth media (Promocell, USA) and the culture components were renewed after 48-72 h. Cell cultures were maintained in CO₂ incubator at 37° C. and 5% CO2. HFDPC were seeded at a density of 20000 cells/well in a 24 well plate in DMEM media with 10% FBS. After the cells reached 80% confluence, induced with testosterone (50 nm) in DMEM media with 2.5% FBS with or without different concentrations of sample for 24 hrs. After treatment period, supernatant was collected for ELISA and the cells were processed for RNA isolation.

### Example 3: Expression of Ornithine-De-Carboxylase (ODC) by Flowcytometry

Ornithine decarboxylase (ODC) enzyme is responsible for a key step in the biosynthesis of polyamines such as spermidine and putrescine. These polyamines have important roles in cell proliferation and blocking them in hair follicles slows the growth of the hair. There is a continual regeneration of ODC. Therefore, when inhibition of this enzyme ceases, the functional activity of ornithine decarboxylase increases (i.e. hair growth resumes)

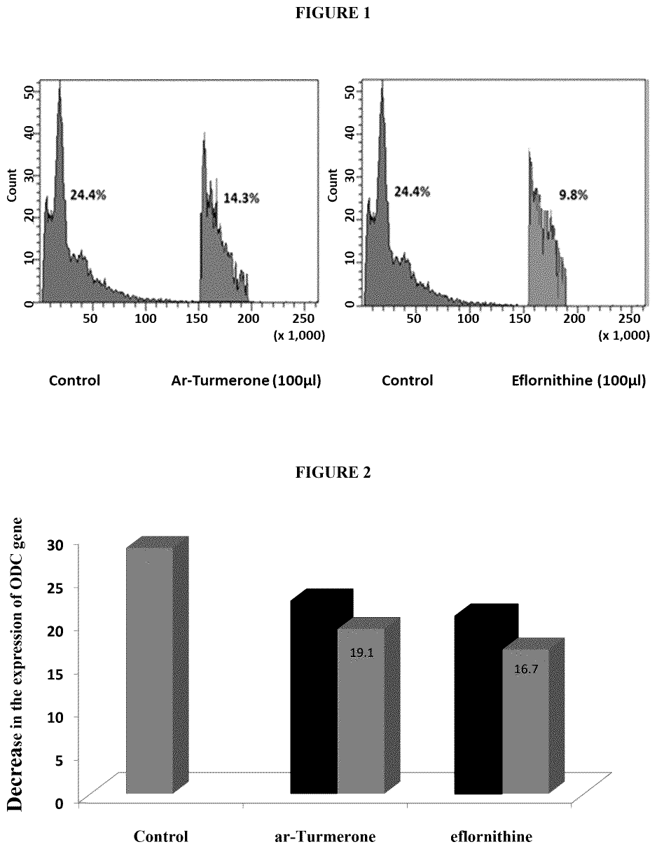

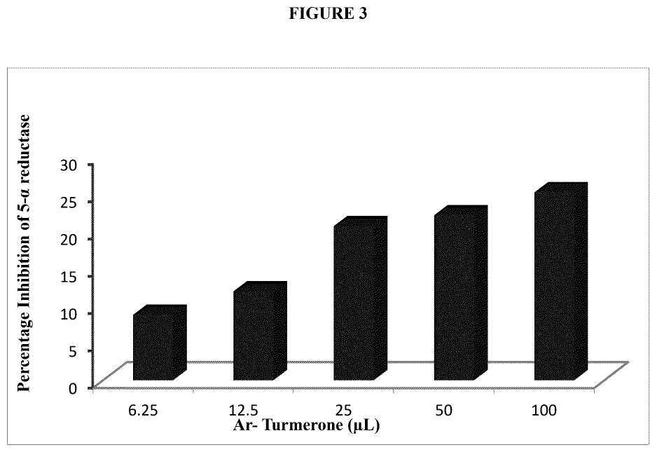

Dermal papilla cells (DPC) were cultured in DMEM medium with 10% FBS and seeded in 96 well tissue culture plate for the test. 24 hrs post seeding, DPC were treated with the test sample Ar-Turmerone at graded concentrations and incubated for 48 hrs in 5% CO₂ incubator at 37° C. After 48 hrs the cells were collected from each well. These cells were incubated with Fluorescein isothiocyanate (FITC) labelled Ornithine-de-carboxylase antibody. After being washed with sterile PBS, samples were resuspended in PBS (pH 7.4) and acquired directly on the Flow cytometer. A fluorescence trigger was set on the FITC (FL1) parameter of the gated DPC populations (10,000 events). Percentage decrease of ODC expression in test samples was calculated with respect to un-treated cells (control), (FIG. 1).

### Example 4: Expression of Ornithine-De-Carboxylase (ODC) by RT-PCR

The total cellular RNA was isolated using Trizol Reagent® (Ambion, Life Technologies, MA, USA), according to the manufacturer's instructions, followed by RNase-free DNase I treatment (Thermo Fisher Scientific) to remove any genomic DNA. Messenger RNA quality and concentration were analyzed spectrophotometrically (Nano Drop Lite, Thermo Fisher Scientific). One microgram of total RNA was reverse transcribed into cDNA using Revert-aid First Strand cDNA synthesis kit (Thermo Fisher Scientific), according to the manufacturer's instructions, and stored at −80° C. until use. Quantitative real-time PCR (qRT-PCR) was performed using SYBR Green qPCR master mix fluorescent dye using Light cycler 96 (Roche Life Science). The primer sequences are given below (Table 1).

TABLE 1
Primer sequences

| Primer | Forward | Reverse |
|---|---|---|
| name | (seq 5′ to 3′) | (seq 5′ to 3′) |
| GAPDH | TGCCACTCAGAAG | TTCAGCTCTGG |
| | ACTGTGG | GATGACCTT |
| ODC-1 | GAGCACATCCCAA | TCCAGAGTCTG |
| | AGCAAAGT | ACGGAAAGTA |

Addition of testosterone increased the ODC expression which was used a master control to compare fold changes from the treatment of active agents. The results are tabulated in table 2.

TABLE 2
Effect of Ar-Turmerone and other extracts
on the Expression of ODC.

| | | Relative mRNA | |
|---|---|---|---|
| | | levels compared | |
| | | to testosterone | Fold |
| Sample | Concentration | treated Control | decrease |
| Untreated | | 0.26243 | |
| Master control | | 1 | 0 |
| (testosterone 50 nm) | | | |
| Ar-Turmerone | 2.5 | 0.29219 | −3.4224 |
| | 5 | 0.38823 | −2.5758 |
| | 10 | 0.47963 | −2.0849 |
| Nigella sativa extract | 10 | 0.53 | −1.886 |
| | 2.5 | 0.471 | −2.12 |
| Oroxylum indicum | 2.5 | 0.2776 | −3.6 |
| extract | | | |
| Licorice extract | 2.5 | — | — |
| Ar-Turmerone + | 2.5 + 2.5 | 0.214 | −4.675 |
| Nigella sativa extract | | | |
| Ar-Turmerone + | 2.5 + 2.5 | 0.288 | −3.47 |
| Licorice extract | | | |
| Ar-Turmerone + | 2.5 + 2.5 + 2.5 | 0.087 | −11.47 |
| Nigella sativa | | | |
| extract + Oroxylum | | | |
| indicum extract | | | |

The results indicated that Ar-Turmerone in combination with Nigella sativa extract and Oroxylum indicum extract showed synergistic activity in inhibiting the express of ornithine decarboxylase enzyme (Table 2). Similarly, the expression of the enzyme decrease with the treatment of Ar-Turmerone and eflornithine individual and in combination (FIG. 2, Table 3)

TABLE 3
Effect of Ar-Turmerone and eflornithine
on the expression of ODC.

| | % Expression of ODC |
|---|---|
| SAMPLES | at 100 μg/ml |
| Control | 26.8% |
| Eflornithine | 11.4% |
| Ar-Turmerone | 17.5% |
| Ar-Turmerone + E (1:0.5) | 13.8% |
| Ar-Turmerone + E (1:1) | 8.6% |
| Ar-Turmerone + E (2:1) | 15.1% |
| Ar-Turmerone + E (3:1) | 19.4% |

### Example 5: Inhibition of 5α-Reductase Activity

Hyperactivity of 5 alpha-reductase in the skin is considered a major mechanism of excessive hair growth in hirsute women with normal levels of serum androgens (idiopathic hirsutism). Inhibition of 5α-reductase causes direct inhibition of conversion of testosterone to 5α-dihydrotestosterone (DHT). Preventing the conversion of testosterone to dihydrotestosterone by inhibiting 5 alpha-reductase activity could thus be the most rational and effective treatment in this condition (Moghetti et al. J Clin Endocrinol Metab. 79 (4) 1994, 1115-21). 5α-reductase converts reduction of steroid double bond in testosterone to its active form (DHT), inhibition yields decreased amounts of DHT and thereby elevation in testosterone and estradiol levels. The quantification of 5α-DHT in the dermal papilla cells using quantitative ELISA is directly proportional to the extent of 5α-reductase inhibition.

In the dermal papilla cells, testosterone exerts its effect after conversion by the enzyme 5α-reductase to the 5 times more potent 5α-dihydrotestosterone (5α-DHT). Inhibition of 5α-reductase thus causes direct inhibition of conversion of testosterone to 5α-dihydrotestosterone. The amount of 5α-DHT produced by the cells was quantified from the supernatant collected, using ELISA method (DHT ELISA KIT, Fine test, Cat #EU2551) as per the manufactures instructions and absorbance was measured at 450 nm using Tecan microplate reader (Table 4).

TABLE 4
Dose Dependent Inhibition of 5α-reductase activity

| | μg/ml | % inhibition |
|---|---|---|
| Ar-Turmerone | 20 | 24.77235 |
| | 10 | 17.77252 |
| | 5 | 9.452557 |
| | 2.5 | 8.559852 |
| Oroxylum indicum extract | 10 | 10.40929 |
| | 5 | 12.94487 |
| Nigella sativa extract | 20 | 23.11642 |
| | 10 | 11.3029 |
| | 5 | 3.028831 |
| Licorice extract | 5 | 14.97997 |
| | 2.5 | 6.228721 |
| Ar-Turmerone + Oroxylum | 10 + 10 | 34.2318 |
| indicum Extract | | |
| Ar-Turmerone + Nigella sativa | 10 + 10 | 38.59479 |
| Extract | | |
| Ar-Turmerone + Licorice | 5 + 2.5 | 24.05241 |
| Extract | | |

Similarly, Eflornithine (E) in combination with Ar-Turmerone (AR-T) also inhibited 5α-reductase activity (Table 5)

TABLE 5
Inhibition of 5 alpha reductase

PERCENTAGE INHIBITION

| | 100 | 50 | 25 | 12.5 |
|---|---|---|---|---|
| SAMPLES | μg/ml | μg/ml | μg/ml | μg/ml |
| Eflornithine | — | — | — | — |
| Ar-Turmerone | 21.34 | 15.24 | 15.03 | 8.04 |
| Ar-Turmerone + E (1:0.5) | 20.61 | 14.66 | 13.53 | 11.34 |
| Ar-Turmerone + E (1:1) | 20.42 | 17.44 | 12.89 | 14.17 |
| Ar-Turmerone + E (2:1) | 18.72 | 15.81 | 13.95 | 11.2 |
| Ar-Turmerone + E (3:1) | 3.04 | 6.02 | 12.62 | 6.87 |

The results indicate that turmeric oil standardized to contain Aromatic Turmerone; Eflornithine; Nigella sativa extract; Oroxylum indicum extract; and Licorice extract either individually or in combination inhibit both ornithine decarboxylase and 5a-reductase and can be used for the management of hirsutism effectively. It is obvious for a person skilled in to art to use the in-vitro results and correlate it effectively for management of hirsutism in a mammal.

### Example 6: Formulations

Tables 6-10 provide illustrative examples of different formulations containing the active ingredients

TABLE 6
Ar-Turmerone cream

Active Ingredients
Turmeric oil standardized to contain Ar-Turmerone
Excipients
Purified Water, Disodium EDTA, Carbopol Ultrez 10, Arlacel 2121,
Phenoxyethanol, Sodium Hydroxide Pellets, Neelicol Tartrazine,
Fragrance: Rich Flora

TABLE 7
Eflornithine cream

Active Ingredients
Eflornithine
Excipients
Purified Water, Disodium EDTA, Carbopol Ultrez 10, Arlacel 2121,
Phenoxyethanol, Sodium Hydroxide Pellets, Neelicol Tartrazine,
Fragrance: Rich Flora

TABLE 8
Nigella sativa cream

Active Ingredients
Nigella sativa extract
Excipients
Purified Water, Disodium EDTA, Carbopol Ultrez 10, Arlacel 2121,
Phenoxyethanol, Sodium Hydroxide Pellets, Neelicol Tartrazine,
Fragrance: Rich Flora

TABLE 9
Oroxylum indicum cream

Active Ingredients
Oroxylum indicum extract
Excipients
Purified Water, Disodium EDTA, Carbopol Ultrez 10, Arlacel 2121,
Phenoxyethanol, Sodium Hydroxide Pellets, Neelicol Tartrazine,
Fragrance: Rich Flora

TABLE 10
Formulation cream

Active Ingredients
Turmeric oil standardized to contain Ar-Turmerone
Nigella sativa extract
Oroxylum indicum extract
Excipients
Purified Water, Disodium EDTA, Carbopol Ultrez 10, Arlacel 2121,
Phenoxyethanol, Sodium Hydroxide Pellets, Neelicol Tartrazine,
Fragrance: Rich Flora

The above formulations are merely illustrative examples, any formulation containing the above active ingredient intended for the said purpose will be considered equivalent. Other modifications and variations of the invention will be apparent to those skilled in the art from the foregoing disclosure and teachings. Thus, while only certain embodiments of the invention have been specifically described herein, it will be apparent that numerous modifications may be made thereto without departing from the spirit and scope of the invention and is to be interpreted only in conjunction with the appended claims.

We claim:

1. A method of management of hirsutism in a subject, comprising a) identifying the subject in need of hirsutism management and b) administering a composition comprising turmeric oil standardized to contain Aromatic Turmerone (Ar-Turmerone); Eflornithine; Nigella sativa extract; Oroxylum indicum extract; and Licorice extract; either individually or combinations thereof.

2. The method as in claim 1, wherein the management of hirsutism is brought about by inhibiting enzymes involved in modulating hair growth, thereby decreasing hair growth in the subject.

3. The method as in claim 1, wherein combinations are selected from the group consisting of turmeric oil standardized to contain Ar-Turmerone and Eflornithine, turmeric oil standardized to contain Ar-Turmerone and Nigella sativa extract, turmeric oil standardized to contain Ar-Turmerone and Oroxylum indicum extract, turmeric oil standardized to contain Ar-Turmerone and Licorice extract, or turmeric oil standardized to contain Ar-Turmerone, Oroxylum indicum extract and Nigella sativa extract.

4. The method as in claim 1, wherein the turmeric oil is standardized to contain 20-50% w/w Ar-Turmerone and 50-90% w/w total turmerones.

5. The method as in claim 1, wherein the Nigella sativa extract is standardized to contain not less than 2% w/w thymoquinone, 0.01-10% w/w thymohydroquinone, 15-95% w/w fatty acids, 0.001-3% w/w α-hederin, 0.1-4% w/w rosmarinic acid and 0.2-2% w/w piperine.

6. The method as in claim 1, wherein the Oroxylum indicum extract is standardized to contain 10-15% w/w oroxylin A, 10-25% w/w baicalein, and 2-10% w/w chrysin.

7. The method as in claim 1, wherein the Licorice extract is standardized to contain 40-50% w/w glabridin.

8. The method as in claim 2, wherein the enzymes are selected from the group consisting of ornithine decarboxylase and 5-α-reductase.

9. The method as in claim 3, wherein the individual extracts in the combination are present in amounts of not less than 10% w/w of the total composition.

10. The method as in claim 1, wherein the subject is a mammal.

11. The method as in claim 1, wherein the composition further comprises of stabilizing agents, bioavailability enhancers, antioxidants, cosmeceutically accepted excipients and topically administered in the form of cream, lotion, oil, serum, gel or emulsion.

12. A method of inhibiting enzymes involved in modulating hair growth in a subject, comprising step of bringing into contact a composition comprising turmeric oil standardized to contain Ar-Turmerone; Eflornithine; Nigella sativa extract; Oroxylum indicum extract; and Licorice extract; either individually or combinations thereof with the said subject to bring about reduction in hair growth.

13. The method as in claim 12, wherein the combinations are selected from the group consisting of turmeric oil standardized to contain Ar-Turmerone and Eflornithine, turmeric oil standardized to contain Ar-Turmerone and Nigella sativa extract, turmeric oil standardized to contain Ar-Turmerone and Oroxylum indicum extract; turmeric oil standardized to contain Ar-Turmerone and Licorice or turmeric oil standardized to contain Ar-Turmerone, Oroxylum indicum extract and Nigella sativa extract.

14. The method as in claim 12, wherein the turmeric oil is standardized to contain 20-50% w/w Ar-Turmerone and 50-90% w/w total turmerones.

15. The method as in claim 12, wherein the Nigella sativa extract is standardized to contain not less than 2% w/w Thymoquinone, 0.01-10% w/w Thymohydroquinone, 15-95% w/w fatty acids, 0.001-3% w/w α-hederin, 0.1-4% w/w rosmarinic acid and 0.2-2% w/w piperine.

16. The method as in claim 12, wherein the Oroxylum indicum extract is standardized contain 10-15% w/w oroxylin A, 10-25% w/w Baicalein, and 2-10% Chrysin.

17. The method as in claim 12, wherein the Licorice extract is standardized to contain 40-50% w/w glabridin.

18. The method as in claim 12, wherein the enzymes are selected from the group consisting of Ornithine decarboxylase and 5-α-reductase.

19. The method as in claim 12, wherein inhibiting enzymes involved in hair growth is effective in the management of hirsutism.

20. The method as in claim 13, wherein the individual extracts in the combination are present in amounts of not less than 10% w/w of the total composition.

21. The method as in claim 13, wherein the subject is a mammal or human cell.

22. The method as in claim 13, wherein the composition further comprises of stabilizing agents, bioavailability enhancers, antioxidants, cosmeceutically accepted excipients and topically administered in the form of cream, lotion, oil, serum, gel or emulsion.


SEQUENCE LISTING (4 sequences)

SEQ ID NO: 1 - DNA, 20 bp, synthetic construct
  source 1..20: mol_type = other DNA
tgccactcagaagactgtgg

SEQ ID NO: 2 - DNA, 20 bp, synthetic construct
  source 1..20: mol_type = other DNA
ttcagctctgggatgacctt

SEQ ID NO: 3 - DNA, 21 bp, synthetic construct
  source 1..21: mol_type = other DNA
gagcacatcccaaagcaaagt

SEQ ID NO: 4 - DNA, 21 bp, synthetic construct
  source 1..21: mol_type = other DNA
tccagagtctgacggaaagta